(19) United States
(12) Patent Application Publication
(10) Pub. No.: US 2003/0004132 A1
(43) Pub. Date: Jan. 2, 2003

(54) Method and material for treating immune diseases

(76) Inventors: Yan Chang, Ashland, MA (US); Vodek Sasak, Northboro, MA (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/176,022

(22) Filed: Jun. 20, 2002

Related U.S. Application Data
(60) Provisional application No. 60/300,360, filed on Jun. 22, 2001.

(51) Int. Cl. A61K 31/732

(52) U.S. Cl. 514/54

(57) ABSTRACT

An immune response in an organism is controlled by administering to said organism a therapeutically effective amount of a compound which binds to a galectin. In specific instances the compound is selected to bind to galectin-1 or galectin-3. Some therapeutic materials comprise natural or synthetic polymers having galactose or arabinose terminated side chains pendent therefrom. A group of preferred therapeutic compounds comprise modified pectins or other materials having a substantially demethoxylated rhamnogalacturan backbone having rhamnose residues interrupting the backbone. One therapeutic material includes a first functional portion which binds to the carbohydrate binding portion of a galectin, and a second functional portion which is operable to denature the galectin protein.

### RELATED APPLICATION

This application claims priority of U.S. Provisional Patent Application Serial No. 60/300,360 filed Jun. 22, 2001, entitled “Method and Material for Treating Immune Diseases.”

### FIELD OF THE INVENTION

This invention relates generally to materials and methods for the treatment of disease in animals. More specifically, the invention relates to materials and methods for the treatment of immune-reaction based diseases in animals. Most specifically, the invention relates to methods and materials for the treatment of autoimmune and other immune-reaction based diseases by the use of compounds which bind to galectins.

### BACKGROUND OF THE INVENTION

Medical science is coming to realize that many disease conditions are, at least in part, resultant from aberrant or excessive immune response within an organism. Autoimmune diseases are significant pathologies which are increasing in our population. Autoimmune disease occurs when an organism mounts an inappropriate immunological response against various of its own proteins or other molecules. Many conditions, including glomerular disease, lupus, rheumatoid arthritis, and atherosclerosis are understood to have an autoimmune basis. Other conditions such as allergies, host-graft rejection and graft-hose rejection are also resultant from an inappropriate or excessive immune response.

Galectins comprise a family of proteins which are expressed by plant and animal cells, and which bind β-galactoside sugars. These proteins can be found on cell surfaces, in cytoplasm, and in extra-cellular fluids. They have a molecular weight in the general range of 29-34 Kd; they have an affinity for β-galactoside containing materials, and have been found to play important roles in a number of biological processes. Galectin-1 and galectin-3 are specific members of this family which have been found to interact with a number of cells and molecules of the immune system. Specifically, galectin-3 has been shown to attract and interact with monocytes, macrophages, and species such as the CD8+ receptor. Additionally, high levels of galectins have been found in tissues manifesting rheumatoid arthritis and other immune moderated diseases.

Various therapies have been implemented to control immune based diseases. Some present therapies rely upon the use of steroid compositions or immunosuppressive drugs. These materials are very toxic and often can produce severe side effects, particularly when administered systemically. As a result, many immune based conditions cannot be adequately treated at the present. It will thus be appreciated that there is a need for therapeutic materials and methods which can moderate immune system responses. Furthermore, such materials and methods should have low toxicity and preferably should be easy to implement. The present invention recognizes that galectins play a significant role in moderating immune reactions. The invention further recognizes that compounds which interact with galectins can significantly affect immune reactions. As will be explained in detail hereinbelow, the present invention provides methods and materials which are based upon the use of carbohydrate based compounds which interact with galectins so as to moderate and control various immune responses. These materials are of low toxicity and are effective agents for the control of immune based disease conditions as is explained hereinbelow.

### BRIEF DESCRIPTIONS OF THE INVENTION

Disclosed herein are methods for controlling an immune response in an organism. The methods comprise administering to said organism a therapeutically effective amount of a compound which binds to a galectin. In particular embodiments, the galectin is found on the cell surface of a tissue, and in specific embodiments of the invention, the therapeutic compound binds to a galectin-1 or galectin-3 receptor.

One class of therapeutic materials having utility in the present invention comprise natural or synthetic polymers having one or more side chains dependent therefrom, which side chains are terminated by a galactose or arabinose sugar. A specific class of therapeutic materials comprise substantially demethoxylated polygalacturonic acids which are interrupted with rhamnose residues.

Modified pectin materials are a particularly preferred class of therapeutic materials for the practice of the present invention, with modified citrus pectins being one preferred member of this group. The pectins are modified by chemical, thermal or enzymatic methods which decrease the chain length of the backbone of the pectin and decrease the branching of side chains thereon. Another group of therapeutic materials of the present invention includes a first functional portion which binds to the carbohydrate binding site of a galectin, and a second functional portion which is operable to denature a protein.

The materials of the present invention can be administered orally, by injection, topically or transdermally.

### THE PRESENT INVENTION

The present invention recognizes the role of galectins in autoimmune diseases, and provides a therapeutic material which will advantageously interact with galectins so as to moderate or prevent the manifestations of immune disease. Specifically, the present invention recognizes that particular carbohydrate materials will bind to galectins and thereby modify their interaction with monocytes, macrophages, and other species which mediate unwanted immune responses.

While galectins are known to bind galactose and other such simple sugars in vitro, those simple sugars are not therapeutically effective in moderating immune system responses in vivo. While not wishing to be bound by speculation, the inventors hereof presume that such relatively small sugar molecules are incapable of blocking, activating, suppressing, or otherwise interacting with other portions of the galectin protein. Therefore, preferred materials for the practice of the present invention generally comprise molecules which contain an active galectin binding sugar site, but which have somewhat higher molecular weights than simple sugars. Such molecules preferably have a minimum molecular weight of at least 300 daltons, and most typically a minimum molecular weight in the range of 300-2,000 daltons. Some specifically preferred materials have yet higher molecular weight ranges. A preferred class of therapeutic materials comprises oligomeric or polymeric species having one or more sugars such as galactose or arabinose pendent therefrom. The oligomeric or polymeric backbone may be synthetic or organic. Materials of this type are disclosed in U.S. Pat. No. ______ (EX Ser. No. 09/750,726) the disclosure of which is incorporated herein by reference. Such materials will preferably have a molecular weight in the range of 300-50,000 daltons. It should be kept in mind that there is some inherent uncertainty in molecular weight measurements of high molecular weight carbohydrates, and measured molecular weights will be somewhat dependent on the method used for measuring the molecular weight. Molecular weights given herein are based on viscosity measurements, and such techniques are known in the art.

One group of materials falling within this general class comprises a substantially demethoxylated polygalacturonic acid backbone having rhamnose residues pendent therefrom. It is believed that in materials of this type, the terminal galactose or arabinose units pendent from the backbone bind to galectin proteins. The remaining bulk of the molecule potentiates the compound's action in moderating immune system response, and as discussed hereinabove, the inventors, while not wishing to be bound by speculation, believe that the remaining bulk of the molecule either interacts with remaining portions of the galectin protein and/or prolongs the binding of the sugar portion thereto. Materials of this general type are described by formulas I, II and III hereinbelow, and it is to be understood that yet other variants of this general compound may be prepared and utilized in accord with the principles of the present invention.

$$\begin{matrix}- {[ α - D - GalpA - ( 1 -> 4 ) - α - D - GalpA ]}_{n} - \\ ↑ \\ {X}_{n - 1} \\ ↑ \\ α - L - Arap \\ \begin{matrix}- {[ α - D - GalpA - ( 1 -> 4 ) - α - D - GalpA ]}_{n} - \\ ↑ \\ {X}_{n - 1} \\ ↑ \\ β - D - Galp\end{matrix}\end{matrix} \qquad ( I )$$

wheren>1.

$$\begin{matrix}β - D - Galp \\ ↓ \\ {X}_{n - 1} \\ ↓ \\ - {[ α - L - Rhap - ( 1 \to 4 ) - α - D - GalpA - ( 1 \to 2 ) ]}_{n} -\end{matrix} \qquad ( II )$$

wheren>1.

$$\begin{matrix}α - L - Arap \\ ↓ \\ {X}_{n - 1} \\ ↓ \\ - {[ α - L - Rhap - ( 1 \to 4 ) - α - D - GalpA - ( 1 \to 2 ) ]}_{n} -\end{matrix} \qquad ( III )$$

where n>1.

Pectin is a complex carbohydrate having a highly branched structure comprised of a polygalacturonic backbone with numerous branching side chains dependent therefrom. The branching creates regions which are characterized as being “smooth” and “hairy.” It has been found that pectin can be modified by various chemical, enzymatic or physical treatments to break the molecule into smaller portions having a more linearized, substantially demethoxylated polygalacturonic backbone with pendent side chains of rhamnose residues having decreased branching. This material is known in the art as modified pectin, and its efficacy in treating cancer has been established. U.S. Pat. No. 5,895,784, the disclosure of which is incorporated herein by reference, describes modified pectin materials, techniques for their preparation, and use of the material as a treatment for various cancers. The material of the '784 patent is described as being prepared by a pH based modification procedure in which the pectin is put into solution and exposed to a series of programmed changes in pH which results in the breakdown of the molecule to yield therapeutically effective modified pectin. The material in the '784 patent is most preferably prepared from citrus pectin; although, it is to be understood that modified pectins may be prepared from pectin starting material obtained from other sources, such as apple pectin and the like. Also, modification processes may be accomplished by enzymatic treatment of the pectin, or by physical processes such as heating. Further disclosure of modified pectins and techniques for their preparation and use are also disclosed in U.S. Pat. No. 5,834,442 and U.S. patent application Ser. No. 08/024,487, the disclosures of which are incorporated herein by reference. Modified pectins of this type generally have molecular weights in the range of 1-50 kilodalton, and a preferred group of such materials has an average molecular weight of about 1-15 kilodalton, and one specific group of materials has a molecular weight of approximately 10 kilodalton.

As disclosed in the prior art, such modified pectin materials have therapeutic efficacy against a variety of cancers. These materials interact with galectins, including galectin-1 and galectin-3, and in that regard also have efficacy against immune based diseases. In accord with the present invention, autoimmune diseases can be controlled or moderated by the use of modified pectin materials and other materials which interact with galectins. These materials may be administered orally; or by intravenous injection; or by injection directly into an affected tissue, as for example by injection into an arthritic joint. In some instances the materials may be administered topically, as in the form of eye drops, nasal sprays, ointments or the like. Also, other techniques such as transdermal delivery systems, inhalation or the like may be employed.

While the foregoing discussion has been primarily directed to therapeutic materials based upon modified pectins, it is to be understood that the present invention is not so limited. In accord with the general principles of the present invention, any member of the broad class of compounds which can interact with and block galectins may be employed to treat immune moderated diseases. These materials, in a preferred embodiment, comprise carbohydrate materials, since such materials are low in toxicity and exhibit strong interaction with galectins. Modified pectin materials comprise one particularly preferred group of carbohydrate materials. Likewise, synthetic and semi-synthetic analogs thereof such as polygalacturonic acid materials may be similarly employed.

Yet another class of materials of the present invention comprises molecules which have a first portion, which is typically a carbohydrate, and which is capable of binding to galectins, joined to a second portion which inactivates or otherwise moderates the activity of a protein. This second portion need not be a carbohydrate and can comprise a material which cross links or otherwise denatures the segment of protein comprising an active portion of the galectin protein, or an active portion of another protein which interacts with the galectin. Such materials include active species such as sulfur or other chalcogen elements alone or in combination such as thiols, sulfhydryls and the like. Other active species may comprise cyano groups, thiocyanates, alkylating agents, aldehydes and the like.

It is to be understood that the foregoing discussion and description is illustrative of particular embodiments of the invention, but is not meant to be a limitation upon the practice thereof. It is the following claims, including all equivalents, which define the scope of the invention.

1. A method for controlling an immune response in an organism, said method comprising:
administering to said organism a therapeutically effective amount of a compound which binds to a galectin.

2. The method of claim 1, wherein said galectin is present on the cell surface of a tissue of said organism.

3. The method of claim 1, wherein said compound binds to galectin-1 or galectin-3.

4. The method of claim 1, wherein said compound comprises a substantially demethoxylated polygalacturonic acid which is interrupted with rhamnose residues.

5. The method of claim 1, wherein said compound comprises a polymeric backbone having side chains dependent therefrom, said side chains being terminated by a galactose or arabinose unit.

6. The method of claim 1, wherein said compound comprises a modified pectin.

7. The method of claim 6, wherein said modified pectin comprises a pH modified pectin.

8. The method of claim 6, wherein said modified pectin comprises an enzymatically modified pectin.

9. The method of claim 6, wherein said modified pectin comprises a thermally modified pectin.

10. The method of claim 6, wherein said modified pectin comprises a modified citrus pectin.

11. The method of claim 1, wherein said compound has a molecular weight of at least 300 dalton.

12. The method of claim 1, wherein said compound has a molecular weight in the range of 300-2,000 dalton.

13. The method of claim 6, wherein said modified pectin has a molecular weight in the range of 1-50 kilodalton.

14. The method of claim 6, wherein said modified pectin has a molecular weight in the range of 1-15 kilodalton.

15. The method of claim 6, wherein said modified pectin has a molecular weight of approximately 10 kilodalton.

16. The method of claim 1, wherein said step of administering said compound to said organism comprises injecting said compound into said organism.

17. The method of claim 1, wherein said step of administering said compound to said organism comprises topically applying said compound to said organism.

18. The method of claim 1, wherein said step of administering said compound to said organism comprises administering said compound transdermally.

19. The method of claim 1, wherein the step of administering said compound to said organism comprises orally administering said compound.

20. A method for the therapeutic treatment of an autoimmune disease in an animal, said method comprising:
administering to said animal a therapeutically effective amount of a compound which binds to a galectin.

21. The method of claim 20, wherein said galectin is present on the cell surface of a tissue of said animal.

22. The method of claim 20, wherein said compound binds to galectin-1 or galectin-3.

23. The method of claim 20, wherein said compound comprises a substantially demethoxylated polygalacturonic acid which is interrupted with rhamnose residues.

24. The method of claim 20, wherein said compound comprises a polymeric backbone having side chains dependent therefrom, said side chains being terminated by a galactose or arabinose unit.

25. The method of claim 20, wherein said compound comprises a modified pectin.

26. The method of claim 25, wherein said modified pectin comprises a pH modified pectin.

27. The method of claim 25, wherein said modified pectin comprises an enzymatically modified pectin.

28. The method of claim 25, wherein said modified pectin comprises a thermally modified pectin.

29. The method of claim 25, wherein said modified pectin comprises a modified citrus pectin.

30. The method of claim 20, wherein said compound has a molecular weight of at least 300 dalton.

31. The method of claim 20, wherein said compound has a molecular weight in the range of 300-2,000 dalton.

32. The method of claim 25, wherein said modified pectin has a molecular weight in the range of 1-50 kilodalton.

33. The method of claim 25, wherein said modified pectin has a molecular weight in the range of 1-15 kilodalton.

34. The method of claim 25, wherein said modified pectin has a molecular weight of approximately 10 kilodalton.

35. The method of claim 20, wherein said step of administering said compound to said animal comprises injecting said compound into said animal.

36. The method of claim 20, wherein said step of administering said compound to said animal comprises topically applying said compound to said animal.

37. The method of claim 20, wherein said step of administering said compound to said animal comprises administering said compound transdermally.

38. The method of claim 20, wherein the step of administering said compound to said animal comprises orally administering said compound.

39. The method of claim 20, wherein said autoimmune disease comprises rheumatoid arthritis.

40. The method of claim 20, wherein said autoimmune disease comprises atherosclerosis.

41. The method of claim 20, wherein said autoimmune disease comprises a glomerular disease.

42. A method for treating an autoimmune disease in an animal, said method comprising:
administering to said animal a compound which binds to a galectin whereby said compound blocks binding of biogenic, immune response invoking, compounds to said galectin.

43. A compound for controlling an immune response in an animal, said compound comprising:
a first functional portion operable to bind to the carbohydrate binding site of a galectin, said first functional portion including a terminal galactose or arabinose; and
a second functional portion operable to denature a protein, said second functional portion including a member selected from the group consisting of chalcogen elements, thiols, sulfhydryls, cyano groups, thiocyanates, alkylating agents, and combinations thereof.

44. The compound of claim 43, wherein said first and second functional portions are attached to a polymeric or oligomeric backbone.
